(19) United States
(12) Patent Application Publication
(10) Pub. No.: US 2024/0336551 A1
(43) Pub. Date: Oct. 10, 2024

(54) BIODEGRADEBLE MATERIAL MANUFACTURING METHOD AND SYSTEM

(71) Applicant: NOROO IC Co., Ltd., Suwanee, GA (US)

(72) Inventor: Hang Duk Roh, Hwaseong-si (KR)

(21) Appl. No.: 18/628,067

(22) Filed: Apr. 5, 2024

(30) Foreign Application Priority Data
Apr. 7, 2023 (KR) 10-2023-0045961

(51) Int. Cl. C07C 51/10; C01B 3/34; C07C 57/04

(52) U.S. Cl. CPC C07C 51/10; C01B 3/346; C07C 57/04; C01B 2203/0233; C01B 2203/06; C01B 2203/1241

(57) ABSTRACT

Disclosed is a system for manufacturing a biodegradable material by utilizing a reforming material of an energy raw material. Specifically, the present invention relates to a system and a business model capable of building a carbon circulation ecosystem by building a system for utilizing carbon monoxide generated in a process of producing hydrogen by using natural gas as a carbon source of a biodegradable material, and provides a system for manufacturing a biodegradable material includes a first system (100) for performing a process of producing hydrogen and carbon monoxide by using an energy raw material, a second system (200) for performing a process of producing an intermediate by using carbon of the carbon monoxide produced in the first system (100) as a carbon source, and a third system (300) for performing a process of manufacturing a biodegradable material by using the intermediate.

### CROSS-REFERENCE TO RELATED APPLICATION

This application claims priority to Korean Patent Application No. 10-2023-0045961 filed on Apr. 7, 2023, and all the benefits accruing therefrom under 35 U.S.C. § 119, the contents of which are incorporated by reference in their entirety.

BACKGROUND

The present invention relates to a method and a system for manufacturing a biodegradable material by utilizing a reforming material of an energy raw material, and specifically, to a method and a system capable of building a carbon circulation ecosystem by building a method and a system for utilizing carbon of carbon monoxide generated in a process of producing hydrogen by using natural gas as a carbon source of a biodegradable material.

The importance of the energy industry is increasing around the world, and most of all, the demand for hydrogen energy is expected to increase rapidly not only as a raw material for the conventional petrochemical industry, but also in connection with new and renewable energy, especially fuel cells. In the long term, the production of hydrogen obtained from water electrolysis using new and renewable energy and the production of hydrogen by using nuclear energy are attracting attention, but until stable technology is secured, the production of hydrogen by using fossil fuels is analyzed to be the most economical method among large-scale hydrogen production methods.

For example, currently, most hydrogen is produced by a natural gas reforming method, which extracts hydrogen from natural gas, one of the fossil fuels, that is, by reacting methane (CH₄), which is the main component of natural gas, with high-temperature water vapor. However, a method for producing hydrogen through the natural gas reforming method is inexpensive, but has the problem of generating greenhouse gases containing carbon monoxide (CO), a gas that is highly reactive with other gases and is harmful to humans, as a by-product (see Equation 1 below).

CH₄+H₂O→CO+3H₂ [Reaction Equation 1]

Carbon monoxide generated during the production of hydrogen through the natural

gas reforming method reacts with other gases to generate smog, ozone, etc., which are toxic substances that harm the human body. Therefore, in order to process carbon monoxide, which is a by-product of a natural gas reforming reaction, a method for capturing and then heating carbon monoxide to convert the carbon monoxide into carbon dioxide and emitting the carbon dioxide is mainly used, and the carbon dioxide generated at this time is also problematic because it is one of the greenhouse gases which are the main cause of global warming.

Although there is a growing interest in hydrogen energy as an alternative to low carbon and decarbonization, the eco-friendliness of hydrogen is not guaranteed considering the hydrogen production technology that necessarily involves the generation of carbon monoxide, and it is questionable whether various hydrogen economy-related policies are environmentally friendly strategies.

In response to this, green hydrogen, which is produced by electrolyzing water (water electrolysis) with electricity from renewable energy sources such as solar and wind power, is classified as an ideal hydrogen energy because it does not emit any carbon monoxide during the production process, but it is expected to take time to be commercialized due to the high production unit cost thereof.

Meanwhile, petrochemical-based solvents or plastics are used as indispensable materials throughout the industry and in daily life. However, due to the toxicity of petrochemical-based solvents themselves and the low recyclability of petrochemical-based plastics, the use of petrochemical-based solvents or plastics raises a number of social or environmental problems.

Particularly, among most discarded solvents or plastics, those having high durability and non-degradability are accumulated in the environment or absorbed by living organisms, resulting in causing diseases, and some garbage flowing into the sea threatens the lives of marine life and destroys the ecosystem.

In order to solve the problems caused by petrochemical-based solvents or plastics, the development of eco-friendly or biodegradable materials has been actively conducted.

Among eco-friendly materials, materials that decompose by themselves in the living body or in the external environment according to various environmental factors such as moisture, microorganisms, and temperature, and are discharged, absorbed outside the human body, or absorbed as organic matters into the land and the like are referred to as biodegradable materials.

Among biodegradable materials, 3-hydroxy propionate (3-HP) having three carbons and two different functional groups, and a derivative compound thereof such as ethyl 3-hydroxypropionate (ethyl 3-HP), are not only non-toxic and have excellent biodegradable properties, but also have similar physicochemical properties to those of major petrochemical-based solvents typically used, and thus, are expected to be widely used as eco-friendly materials. In addition, poly (3-hydroxypropionate (poly 3-HP) polymerized from 3-hydroxypropionate has excellent biodegradable properties, and thus, is expected to be used in various fields such as medical and general household products and industrial solvents.

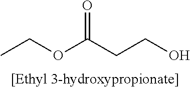

However, until now, the production of 3-HP and 3-HP derivative compounds by a petrochemical-based manufacturing method has been complicated and has had very low efficiency, so that currently, only a biological production process (e.g., a fermentation process) is proposed as an alternative. However, due to the high production unit cost thereof, the biological production process has been an obstacle to commercialization of 3-HP and 3-HP derivative compounds.

### SUMMARY

The present disclosure provides a method, a system, and a business model for building a carbon circulation ecosystem as a method and a system for simultaneously solving the above-described problems, and specifically, provides a convergence business model capable of breaking down the boundaries between the energy industry and the chemical industry by building a method and a system for utilizing carbon of carbon monoxide generated in a step of reforming an energy raw material as a carbon source of a biodegradable material.

The present disclosure also provides a method and a system capable of building a carbon circulation ecosystem by utilizing carbon of carbon monoxide as a carbon source of a biodegradable material, thereby reducing the emission of substances that cause global warming.

The present disclosure also provides a method and a system that are advantageous from the perspective of resource circulation or carbon circulation by humankind.

Other objects, specific advantages, and novel features of the present invention will become more apparent from the following detailed description and preferred embodiments in connection with the accompanying drawings.

In accordance with an exemplary embodiment of the present invention, a system for manufacturing a biodegradable material includes a first system 100 for performing a process of producing hydrogen and carbon monoxide by using an energy raw material, a second system 200 for performing a process of producing an intermediate by using carbon of the carbon monoxide produced in the first system 100 as a carbon source, and a third system 300 for performing a process of manufacturing a biodegradable material by using the intermediate.

In an embodiment of the present invention, the process of producing hydrogen and carbon monoxide by using an energy raw material may use a reforming reaction.

In an embodiment of the present invention, the energy raw material may be any one selected from natural gas, LPG, and SYN GAS.

In an embodiment of the present invention, the second system 200 may produce an intermediate by performing a catalyst-carbonylation reaction on a stream containing the carbon monoxide produced in the first system 100.

In an embodiment of the present invention, the second system 200 may produce an intermediate by performing a catalyst-carbonylation reaction on a stream containing the carbon monoxide produced in the first system 100 and a stream containing an epoxide-based compound.

In an embodiment of the present invention, the intermediate may be one or more β-lactone series compounds selected from the group consisting of β-Propio lactone and β-Butyro lactone.

In an embodiment of the present invention, the biodegradable material manufactured in the third system 300 may be 3-hydroxy propionate, 3-hydroxy butyrate, or a derivative thereof.

In an embodiment of the present invention, the derivative may be a copolymer of alkyl 3-hydroxypropionat, poly (3-hydroxypropionate), or 3-hydroxypropionate (here, the alkyl group is an alkyl group having 1 to 5 carbon atoms.), which is highly biodegradable.

In an embodiment of the present invention, the system may further include a system for producing acrylic acid or acrylonitrile by using the 3-hydroxy propionate and a derivative thereof.

In accordance with another exemplary embodiment of the present invention, there is provided a biodegradable material manufactured by the above-described system for manufacturing a biodegradable material.

The above means for achieving the purposes do not include all the features of the present invention. Various features of the present invention and advantages and effects in accordance thereto may be understood in more detail with reference to the following specific embodiments.

### BRIEF DESCRIPTION OF THE DRAWINGS

Exemplary embodiments can be understood in more detail from the following description taken in conjunction with the accompanying drawings, in which:

FIG. 1A is a conceptual diagram showing an entire system 10 of the present invention including a first system 100, a second system 200, and a third system 300;

FIG. 1B is a conceptual diagram showing that the entire system 10 of the present invention including the first system 100, the second system 200, and the third system 300 relates to a comprehensive system which comprehensively includes the energy industry, the chemical industry, and the biochemical industry;

FIG. 2A is a conceptual diagram of the first system 100 showing a step of producing hydrogen energy by steam reforming natural gas, which is an energy raw material;

FIG. 2B is a conceptual diagram of the first system 100 showing a step of producing hydrogen energy by dry reforming natural gas, which is an energy raw material;

FIG. 3 is a conceptual diagram showing a step of producing carbon dioxide through a conversion reaction following a reforming reaction of natural gas; and

FIG. 4 shows the degree of biodegradability of ethyl 3-HP, 3-HP, and Poly (3-HP).

### DETAILED DESCRIPTION OF EMBODIMENTS

Hereinafter, the present invention will be described in more detail with reference to the accompanying drawings. However, the accompanying drawings are merely examples for easily describing the contents and scope of the technical idea of the present invention, and the technical scope of the present invention is not limited or changed thereby. In addition, it will be obvious to those skilled in the art that various modifications and changes are possible based on these examples within the scope of the technical idea of the present invention.

In addition, it will be understood that terms or words used in the present specification and claims shall not be construed as being limited to having meanings defined in commonly used dictionaries, but should be interpreted as having meanings and concepts consistent with the technical idea of the present invention based on the principle that an inventor may appropriately define concepts of the terms to best explain the invention. Therefore, the embodiment described herein is merely the most preferred embodiment of the present invention, and is not intended to limit the technical idea of the present invention, and therefore, it should be understood that there may be various equivalents and modifications that may substitute the embodiment at the time of the present application.

In describing the present invention, “reforming” refers to a method for converting the form of a chemical structure in the component of a material and synthesizing and extracting a desired material in the process thereof, and in the present invention, mainly refers to a method for extracting hydrogen using a hydrocarbon material contained in natural gas.

In describing the present invention, “biodegradable” refers to a definition based on ASTM D5338-15 (Standard Test Method for Determination of Aerobic Biodegradation of Plastic Materials Under Controlled Composting Conditions, Incorporating Thermophilic Temperatures).

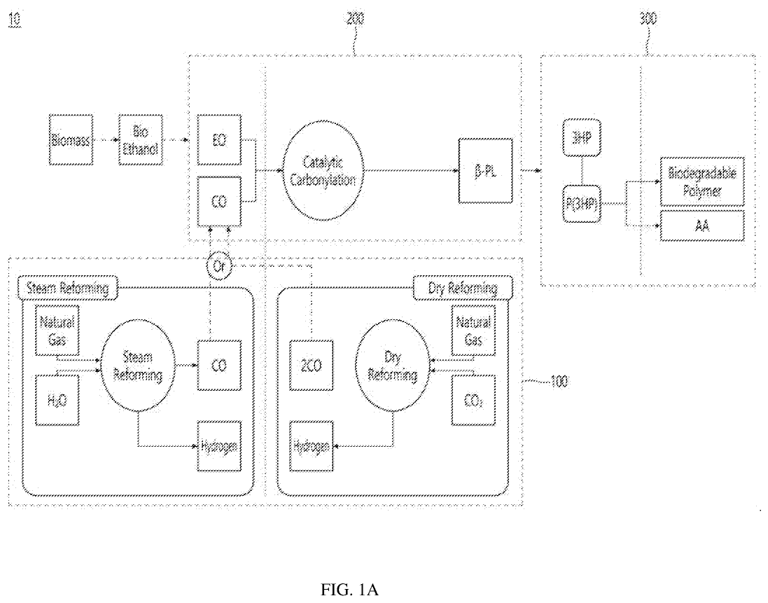

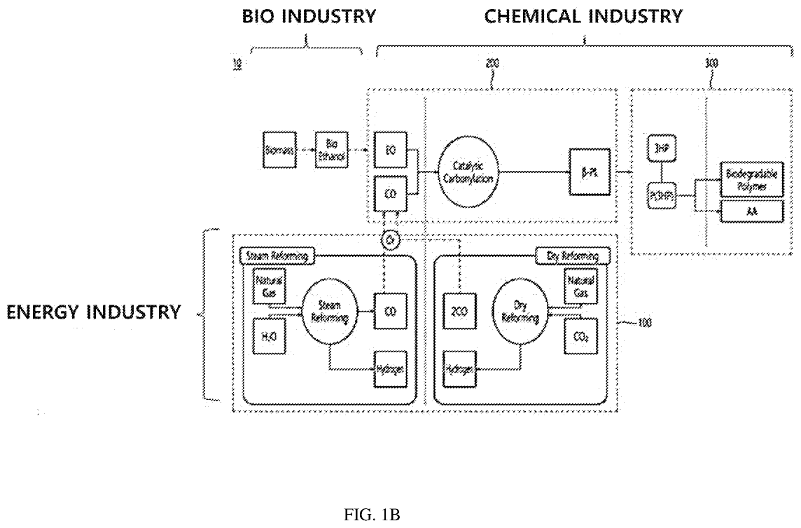

Referring to FIG. 1, a system of the present invention may be composed of three main steps. Specifically, the system of the present invention may provide a system for manufacturing a biodegradable material including a first system 100 for performing a step of producing hydrogen and carbon monoxide by using an energy raw material, a second system 200 for performing a step of producing an intermediate by using carbon of the carbon monoxide produced in the first system 100 as a carbon source, and a third system 300 for manufacturing a biodegradable material by using the intermediate.

As described above, the demand for hydrogen energy is expected to increase rapidly not only as a raw material for the conventional petrochemical industry, but also in connection with fuel cells. Currently, a method for producing hydrogen by steam reforming using natural gas among fossil fuels is adopted as a commercial process, but there is a problem in that carbon monoxide is produced as a by-product, so that Carbon Capture & Storage (CCS), which separates and stores carbon monoxide, and Carbon Capture & Utilization (CCU), which captures and utilizes the same, are required to be additionally performed. Recently, in Korea, a bill to promote the development of the above-described CCS and CCU-related industries is scheduled to be proposed in 2023, and systems for carbon reduction are being supported, and carbon reduction is becoming an important topic.

Accordingly, the present invention is to provide a method and a system for utilizing a step of reforming an energy raw material and carbon of carbon monoxide produced in the step of reforming an energy raw material and as a carbon source of a biodegradable material.

The method and the system of the present invention use carbon of carbon monoxide generated as a by-product in a step of producing hydrogen energy as a carbon source of a biodegradable material, and thus, may reduce the emission of carbon monoxide and manufacture a biodegradable material at low cost, and has an effect of providing a carbon circulation ecosystem.

Hereinafter, a reaction performed in each system will be described in detail.

1. First system 100 for performing process of producing hydrogen and carbon

monoxide by using energy raw material

The first system 100 of the present invention may perform a process of producing hydrogen and carbon monoxide by using an energy raw material. At this time, the energy raw material may be a hydrocarbon composed of carbon and hydrogen, and specifically may include one or more energy raw materials selected from natural gas, liquefied petroleum gas (LPG), and synthetic gas (Syn gas).

In an embodiment of the present invention, the process of producing hydrogen and carbon monoxide by using an energy raw material in the first system 100 may use a reforming reaction.

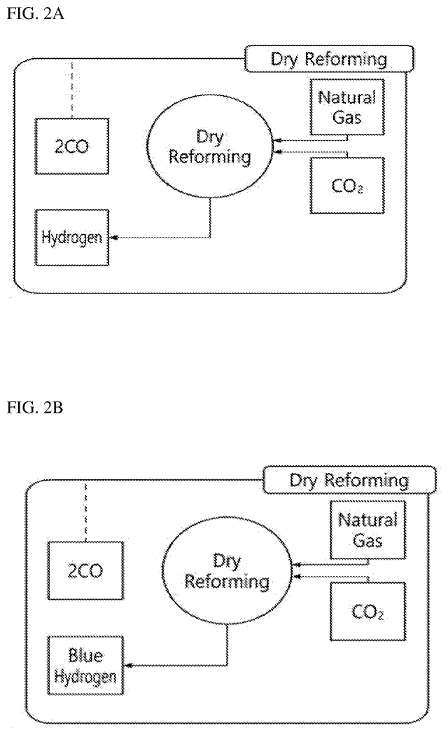

FIG. 2A and FIG. 2B are according to an embodiment of the present invention and are conceptual diagrams of the first system 100 for performing a process of producing hydrogen and carbon monoxide by reforming natural gas, which is one of the energy raw materials.

As illustrated in FIG. 2A and FIG. 2B, the first system 100 of the present invention

is supplied with a stream containing an energy raw material, and may perform a process of producing hydrogen and carbon monoxide by using the energy raw material.

In an embodiment of the present invention, when the stream containing an energy raw material is supplied into the first system 100, a process of reforming the energy raw material may be performed to perform a process of producing hydrogen and carbon monoxide.

In an embodiment of the present invention, the reforming step performed in the first system 100 may be steam reforming or dry reforming.

The steam reforming is a method for supplying energy raw materials, such as natural gas and steam, as reactants. The dry reforming is a method for supplying energy raw materials, such as natural gas and carbon dioxide, as reactants.

In a steam reforming method, which is one of the embodiments of the present invention, a stream containing an energy raw material supplied to the first system 100 and a stream containing steam may be supplied in a ratio typically used in the art, and the ratio is not limited to a specific supply ratio.

A steam reforming process may be performed on energy raw materials, e.g., natural gas and steam to extract hydrogen, and at this time, carbon monoxide is produced as a by-product.

In a dry reforming method, which is another embodiment of the embodiments of the present invention, a stream containing an energy raw material supplied to the first system 100 and a stream containing carbon dioxide supplied to the first system 100 may be supplied in a ratio typically used in the art, and the ratio is not limited to a specific supply ratio.

In the dry reforming method, which is one of the embodiments of the present invention, the temperature of the stream containing an energy raw material may be 200 K to 400 K, and the temperature of the stream containing carbon dioxide may be 500 K to 900 K.

A dry reforming process may be performed on energy raw materials, e.g., natural

gas and carbon dioxide to extract hydrogen, and at this time, carbon monoxide is produced as a by-product.

Meanwhile, among the above-described reforming reactions, the steam reforming is considered to be the cheapest method in hydrogen production, and since hydrogen is produced from both methane and water, it is possible to achieve a high production yield, so the hydrogen production is mostly done using a reforming reaction. Since the steam reforming is the most commonly used hydrogen production method, the amount of carbon monoxide produced as a by-product of the reforming reaction is bound to be very large, and even if CCS and CCU are taken into consideration, there is a need for a method for treating a large amount of carbon monoxide generated as a by-product of the reforming reaction.

The system of the present invention is meaningful in that carbon of a large amount of carbon monoxide generated during the production of hydrogen energy, which is an eco-friendly energy, is utilized as a carbon source of a biodegradable material, thereby reducing carbon monoxide emitted when producing hydrogen energy by using fossil fuels, and furthermore greenhouse gases, which are the main cause of climate change such as global warming, are used in the manufacture of a biodegradable material.

Therefore, according to the system of the present invention, it is possible to prevent global warming and build a carbon circulation ecosystem by utilizing carbon of carbon monoxide, a by-product of producing hydrogen energy, which is an eco-friendly energy, as a carbon source of a biodegradable material.

Meanwhile, hydrogen extracted by reacting natural gas with high-temperature and high-pressure steam or carbon dioxide is referred to as gray hydrogen, and hydrogen which reduces carbon emissions by capturing and storing carbon dioxide generated in the process of producing gray hydrogen is referred to as blue hydrogen. In the present invention, hydrogen is produced using natural gas, which is one of energy raw materials, and carbon monoxide, which is a by-product generated at this time, is captured and stored to be utilized in the second system 200 to be describe below, so that the hydrogen may be referred to as blue hydrogen.

The first system 100 may additionally have a separation system for separating hydrogen and carbon monoxide, which are products. The separation system for separating carbon monoxide from hydrogen may use a system known in the art, and is not particularly limited.

The hydrogen separated in the first system 100 may be used as a hydrogen source in various devices which use hydrogen energy as an energy source, such as fuel cells.

A stream containing carbon monoxide separated in the first system 100 may be supplied back to the second system 200.

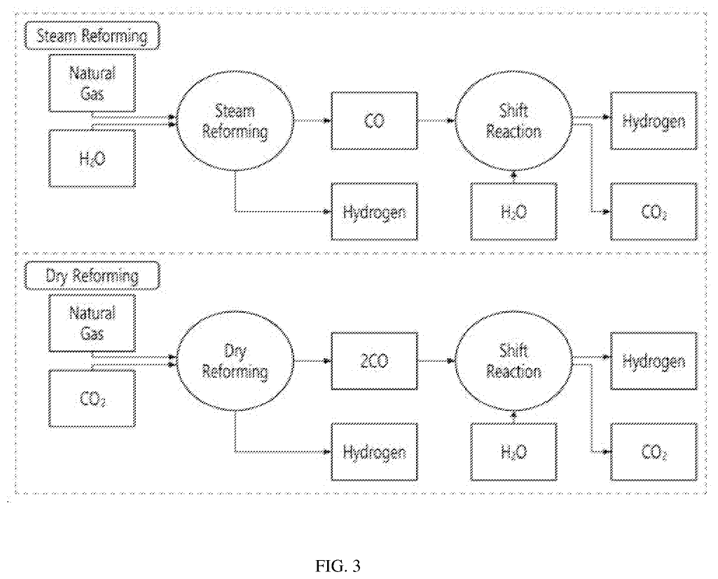

Meanwhile, the first system 100 may further include a system for performing a shift reaction which converts the separated carbon monoxide into carbon dioxide (see FIG. 3).

The conversion reaction is a reaction for producing carbon dioxide by reacting carbon monoxide, a product of the reforming reaction, with steam, and may be set to react a stream containing carbon monoxide with steam if necessary, instead of supplying the stream to the second system 200, to produce carbon dioxide.

2. Second system 200 for producing intermediate, precursor for biodegradable material, by using carbon of carbon monoxide, product of first system 100, as carbon source A stream containing carbon monoxide, a product of the first system 100 and a

stream containing an epoxide-based compound may be supplied as reactants to the second system 200.

In the second system 200, a process of producing an intermediate, which becomes a precursor for a biodegradable material, by subjecting carbon monoxide and an epoxide-based compound to a catalytic reaction is performed.

In describing the present invention, the intermediate, which becomes the precursor for a biodegradable material, is an intermediate compound for manufacturing the biodegradable material, and as to be described below, any compound is sufficient as long as it is a compound that can manufacture a biodegradable material through an open-ring reaction, an open-ring polymerization reaction, or a condensation polymerization reaction followed by opening a ring, and is not particularly limited.

A product utilizing a biodegradable materials, e.g., a plastic product containing a biodegradable polymer, is less price-competitive due to high manufacturing unit cost thereof compared to that of petrochemical-based plastics. However, the present invention uses, as a carbon source of a biodegradable material, carbon (C) contained in carbon monoxide produced as a result of hydrogen production, and thus, has an advantageous effect of lowering the manufacturing unit cost of a product including a biodegradable material.

Particularly, the present invention is technically meaningful in that carbon monoxide emitted as a by-product in a process of producing hydrogen, which can be said to be clean energy, is utilized as a carbon source of a biodegradable material.

In an embodiment of the present invention, the epoxide-based compound is preferably one or more selected from the group consisting of propylene oxide, ethylene oxide, and 1,2-epoxybutane, wherein ethylene oxide is the most preferable, but any compound is sufficient as long as it is a compound that can react with carbon monoxide to produce an intermediate, and the epoxide-based compound is not limited to a specific compound.

Meanwhile, the epoxide-based compound of the present invention may be derived from biomass or bioethanol. Typically, an epoxide-based compound derived from a petrochemical material is used, but since an epoxide-based compound derived from biomass or bioethanol is used as the epoxide-based compound of the present invention, there is an effect of building a carbon circulation ecosystem that utilizes carbon from a bio-derived material as a carbon source of a biodegradable material.

The second system 200 produces an intermediate by performing a catalytic reaction on a stream containing carbon monoxide, which is a product of the first system 100, and a stream containing an epoxide-based compound.

At this time, a catalyst used in the second system 200 is a catalyst known in the art, and any material is sufficient as long as it is a material that can produce an intermediate by performing a catalytic reaction on a stream containing carbon monoxide and a stream containing an epoxide-based compound, and the catalyst is not limited to a specific catalyst.

The stream containing carbon monoxide and the stream containing an epoxide-based compound which are supplied to the second system 200 may be supplied in a ratio typically used in the art, and the ratio is not limited to a specific supply ratio.

In addition, the carbon monoxide and the epoxide-based compound react at a reaction temperature of 500°° C. to 900° C. in the presence of the catalyst to produce an intermediate.

The reaction in the second system 200 is performed while the temperature of the reactor is being maintained at 500° C. to 900° C., and at this time, if the reaction temperature is lower than 500° C., the temperature is too low to provide an energy sufficient enough to proceed with a chemical reaction, so that it is not possible to expect sufficient catalytic activity, and if the reaction temperature is higher than 900° C., it is not preferable because a deactivation phenomenon occurs due to a sintering phenomenon and the like of nickel in the catalyst which is an active phase at high temperatures.

In an embodiment of the present invention, the carbon monoxide and the epoxide-based compound supplied to the second system 200 may be converted into β-Propio lactone, an example of intermediates by a catalyst-carbonylation reaction as set forth in [Reaction Equation 2] below.

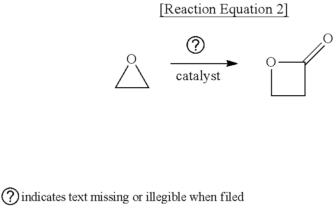

In an embodiment of the present invention, the intermediate produced in the second system 200 may be a β-lactone series compound having 3 to 5 carbon atoms.

For example, the intermediate may be one or more β-lactone series compounds selected from the group consisting of β-Propio lactone and β-Butyro lactone.

3. Third system for manufacturing biodegradable material by using

intermediate produced in second system 200

The third system 300 is a system for manufacturing a biodegradable material by using the intermediate produced in the second system 200.

A stream containing the intermediate produced in the second system 200 may be supplied to the third system 300.

In an embodiment of the present invention, in the third system 300, a biodegradable material is manufactured by an open-ring reaction or open-ring polymerization reaction of the intermediate produced in the second system 200.

The biodegradable material manufactured in the third system 300 may be manufactured by an open-ring reaction and/or polymerization reaction of one or more β-lactone series compounds selected from the group consisting of β-Propio lactone and β-Butyro lactone.

In another embodiment of the present invention, in the third system 300, different derivatives may be polymerized to prepare a co-polymer. For example, the co-polymer may be prepared by open-ring polymerization of a β-Propio lactone and/or β-Butyro lactone co-monomer. A variety of co-monomers may be used with the β-Propio lactone and/or β-Butyro lactone monomer, which may be a co-monomer that add biodegradability to the co-polymer.

In an embodiment of the present invention, the biodegradable material manufactured in the third system 300 may be 3-hydroxy propionate (3-HP), 3-hydroxy butyrate, or a derivative thereof.

For example, a derivative of 3-HP, which is a biodegradable material manufactured in the third system 300, may be alkyl 3-hydroxypropionate in which an alkyl group is substituted in 3-HP, and specifically, may be ethyl 3-hydroxypropionate (here, the alkyl group is an alkyl group having 1 to 5 carbon atoms).

In another example, a biodegradable material manufactured in the third system 300 may be poly 3-hydroxypropionate in which 3-HP is polymerized.

In an embodiment of the present invention, in the third system 300, a blend of the above-described polymer or co-polymer may be prepared.

A series of reactions occurring in the first system 100, the second system 200, and the third system 300 of the present invention use carbon of carbon monoxide as a carbon source to manufacture a biodegradable material. The biodegradable material manufactured in the above manner, e.g., 3-HP, has the same chemical formula structure as that of 3-HP manufactured using a fermentation method, which is a biological manufacturing method. Since 3-HP, which has the same chemical structure formula as that of 3-HP manufactured by a biological method utilizing carbon monoxide generated during hydrogen production, is manufactured, it can be assumed that the physical properties thereof will also be the same. Therefore, the present invention is technically meaningful in that carbon monoxide, which is harmful to the human body and generated during hydrogen production, may be converted into biodegradable and highly versatile 3-HP.

Meanwhile, a biodegradable material manufactured in the system of the present invention is a material that decomposes by itself in the living body or in the external environment and is discharged outside the human body or absorbed as an organic matter into the land and the like, and thus, is harmless due to the biocompatibility thereof, and may become a sustainable material from the environmental point of view, in which lies another technical meaning of the present invention.

A biodegradable material manufactured in the present invention may be applied as an eco-friendly solvent or eco-friendly polymer in various fields.

In an embodiment of the present invention, the present invention may further include a system for performing a process of producing acrylic acid or acrylonitrile by using the 3-hydroxy propionate and a derivatives thereof manufactured in the third system 300. At this time, the process of producing acrylic acid or acrylonitrile by using the 3-hydroxypropionate and the derivative thereof may be performed by using a reaction known in the art.

In an embodiment of the present invention, the third system 300 may use various additives to control the physical properties of a biodegradable material to be finally manufactured.

The additive may include one or more components selected from a flame retardant, a plasticizer, a pigment, heat, a light stabilizer, a filler, and a fiber reinforcing agent.

In an embodiment of the present invention, if a polymerization or copolymerization reaction of an intermediate is performed, it is performed in the presence of a polymerization initiator. The polymerization initiator may prepare a polymer or co-polymer by open-ring polymerization or condensation polymerization after opening a ring for ß-Propio lactone, which is one of intermediates. A polymerization catalyst known in the art may be used for the initiation of open-ring polymerization, and the polymerization catalyst is not particularly limited.

In an embodiment of the present invention, a polymerization initiator may be an ionic initiator. The ionic initiator has the general formula of MX (here, M is cationic and X is anionic). The M is selected from the group consisting of Lit, Na⁺, K⁺, Mg²⁺, Ca²⁺, and Al³⁺. The X is a nucleophilic anion. A suitable nucleophilic anion includes, but is not limited to, a compound composed of at least one carbonoxylate group, at least one alkoxide group, at least one phenoxide group, or a combination thereof.

As such, since a biodegradable material manufactured in the third system 300 has the same chemical structure formula as that of a biodegradable material manufactured by a typical method known in the art, it can be predicted that the biodegradable material manufactured in the third system 300 will have the same physical properties as those of the biodegradable material manufactured by the typical method known in the art.

A biodegradable material manufactured in the system of the present invention may be used in a variety of applications such as solvents, plastics, wrapping paper, and the like.

The first system 100, the second system 200, and the third system 300 of the present invention are meaningful in that a comprehensive system is provided in which carbon monoxide (energy-related reaction), which is a by-product produced as a result of hydrogen production by reforming an energy raw material, is converted into an intermediate through a catalytic reaction (chemical-related reaction), and the resulting intermediate is converted into a biodegradable material (biochemical-related reaction), which is beneficial to the human kind (see FIG. 1B).

Meanwhile, a biodegradable material manufactured through the system of the present invention has the advantage of being able to be recycled or upcycled through hydrolysis or calcination.

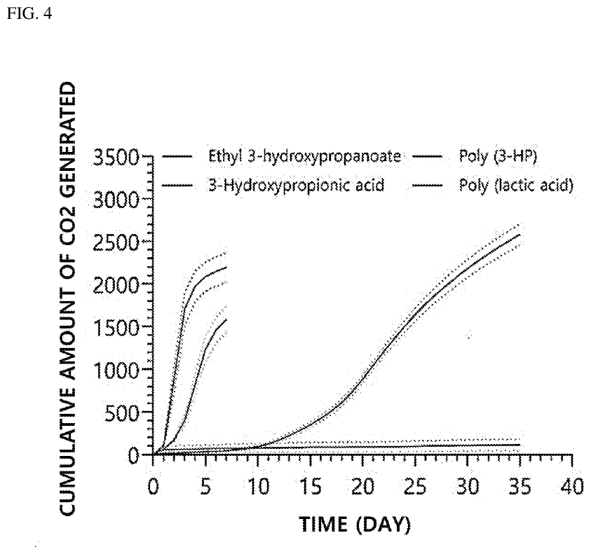

The degree of biodegradability of ethyl 3-HP, 3-HP, and Poly (3-HP), which are biodegradable materials manufactured by the system of the present invention, is shown in FIG. 4.

FIG. 4 is a graph showing the comparison of cumulative amounts of CO₂ generated over time for Ethyl 3-HP, 3-HP, and Poly (3-HP). In the case of ethyl 3-HP and 3-HP, it can be confirmed that the amount of CO₂ generated over time converges to a certain value only in a few days. This is a much faster biodegradation rate than that of poly (lactic acid) (PLA), which is a biodegradable polymer.

In addition, in the case of Poly (3-HP), CO₂ is generated slowly compared to the cases of ethyl 3-HP or 3-HP, but when compared PLA, which is the reference material, it can be confirmed that the amount of CO₂ generated increases much faster. Since PLA generates very little CO₂, it can be assumed that biodegradation hardly occurs in the soil. Therefore, it can be confirmed that 3-HP, ethyl 3-HP, and Poly (3-HP) have excellent biodegradability compared to PLA, which is a biodegradable material.

Meanwhile, a sterilization experiment was conducted on ethyl 3-HP, a biodegradable material manufactured by the system of the present invention, and the data thereof is shown in Table 1 below. The sterilization experiment was conducted by a test method according to the KCL-FIR-1002:2021 standard.

TABLE 1

| | | Initial | Concentra- | Bacteri- |
|---|---|---|---|---|
| | | concen- | tion after | ostatic |
| | | tration | 24 hours | reduction |
| | Samples | (CFU/ml) | (CFU/ml) | rate (%) |
| Antimicrobial test: | BLANK | 1.3*10⁴ | 1.5*10⁵ | |
| Escherichia coli | Ethyl 3- | 1.3*10⁴ | 1.2*10⁵ | 20 |
| (Escherichia coli | HP | | | |
| ATCC 25922) | | | | |
| Antimicrobial test: | BLANK | 3.4*10⁴ | 3.7*10⁵ | |
| Staphylococcus | Ethyl 3- | 3.4*10⁴ | 3.4*10⁵ | 8.1 |
| aureus | HP | | | |
| (Staphylococcus | | | | |
| aureus ATCC 6538) | | | | |
| Antimicrobial test: | BLANK | 2.5*10⁴ | 2.6*10⁵ | |
| MRSA bacteria | Ethyl 3- | 2.5*10⁴ | 1.0*10⁵ | 61.5 |
| (Staphylococcus | HP | | | |
| aureus subsp. | | | | |
| aureus) | | | | |
| Antimicrobial test: | BLANK | 2.6*10⁴ | 2.7*10⁵ | |
| Streptococcus | Ethyl 3- | 2.6*10⁴ | 1.4*10⁵ | 48.1 |
| (Streptococcus | HP | | | |
| mutans ATCC | | | | |
| 25175) | | | | |
| Antimicrobial test: | BLANK | 3.0*10⁴ | 3.1*10⁵ | |
| Streptococcus | Ethyl 3- | 3.0*10⁴ | 1.7*10⁵ | 45.1 |
| (Bacillus cereus | HP | | | |
| ATCC 11778) | | | | |

From the above data, it can be confirmed that 3-HP. 3-HP. and Poly (3-HP) have only the bacteriostatic effect and no sterilizing effect, and therefore, it can be presumed that 3-HP. 3-HP. and Poly (3-HP) are harmless to microorganisms.

According to a method and a system of the present invention, it is possible to build a carbon circulation ecosystem by building a method and a system for utilizing carbon of carbon monoxide generated in a step of reforming an energy raw material as a carbon source of a biodegradable material, and providing the method and the system as a business model.

According to the method and the system of the present invention, it is possible to provide a method and a system that are advantageous from the perspective of resource circulation by humankind by utilizing carbon of carbon monoxide generated during hydrogen production as a carbon source of a biodegradable material, and there is an effect of manufacturing biodegradable materials that can ensure price competitiveness.

A biodegradable material manufactured by the method and the system of the present invention has the same chemical structural formula as that of a biodegradable material manufactured by a biological process, and thus, may be expected to have the same physical properties as the biodegradable material manufactured by the biological process, and furthermore, is harmless to the human body due to the biocompatibility thereof, and may become a sustainable material from the environmental point of view.

In addition to the above-described effects, specific effects of the present invention will be described together while explaining the specific details for carrying out the invention below.

The above-described embodiment of the present invention should not be construed as limiting the technical spirit of the present invention. The scope of protection of the present invention is limited only by the matters stated in the claims, and those skilled in the art may improve and change the technical idea of the present invention into various forms. Therefore, such improvements and changes will fall within the scope of protection of the present invention as long as they are obvious to those skilled in the art.

### [Description of the Reference Numerals or Symbols]

10: Biodegradable material manufacturing system
100: First system
200: Second system
300: Third system

What is claimed is:

1. A system for manufacturing a biodegradable material, the system comprising:
a first system (100) for performing a process of producing hydrogen and carbon monoxide by using an energy raw material;
a second system (200) for performing a process of producing an intermediate by using carbon of the carbon monoxide produced in the first system (100) as a carbon source; and
a third system (300) for performing a process of manufacturing a biodegradable material by using the intermediate.

2. The system of claim 1, wherein the process of producing hydrogen and carbon monoxide by using an energy raw material uses a reforming reaction.

3. The system of claim 2, wherein the reforming reaction is a steam reforming reaction or a dry reforming reaction.

4. The system of claim 1, wherein the energy raw material is any one selected from natural gas, LPG, and SYN GAS.

5. The system of claim 1, wherein the second system (200) produces an intermediate by performing a catalyst-carbonylation reaction on a stream containing the carbon monoxide produced in the first system (100).

6. The system of claim 5, wherein the second system (200) produces an intermediate by performing a catalyst-carbonylation reaction on a stream containing the carbon monoxide produced in the first system (100) and a stream containing an epoxide-based compound.

7. The system of claim 6, wherein the epoxide-based compound is derived from biomass or bioethanol.

8. The system of claim 1, wherein the intermediate is β-lactone series compound having 3 to 5 carbon atoms.

9. The system of claim 8, wherein the intermediate is one or more β-lactone series compounds selected from the group consisting of β-Propio lactone and β-Butyro lactone.

10. The system of claim 1, wherein the biodegradable material manufactured in the third system is 3-hydroxy propionate or a derivative thereof.

11. The system of claim 10, wherein the derivative is a copolymer of alkyl 3-hydroxypropionat, poly (3-hydroxypropionate), or 3-hydroxypropionate (here, the alkyl group is an alkyl group having 1 to 5 carbon atoms.).

12. The system of claim 10, further comprising a system for producing acrylic acid or acrylonitrile by using the 3-hydroxy propionate and a derivative thereof.

13. A biodegradable material manufactured by the system of claim 1.
